(19) United States
(12) Patent Application Publication
(10) Pub. No.: US 2018/0263463 A1
(43) Pub. Date: Sep. 20, 2018

(54) DEVICE FOR OPERATING AN INTRAORAL SCANNER

(71) Applicant: a.tron3d GmbH, Klagenfurt am Woerthersee (AT)

(72) Inventor: Juergen JESENKO, Riegersburg (AT)

(21) Appl. No.: 15/917,918

(22) Filed: Mar. 12, 2018

(30) Foreign Application Priority Data
Mar. 17, 2017 (EP) 17 161 599.0

(51) Int. Cl. A61B 1/00; A61B 1/24; A61G 15/10

(52) U.S. Cl. CPC A61B 1/00032; A61G 15/10; A61B 1/24; A61B 1/00013

(57) ABSTRACT

In a device for operating an intraoral scanner with at least one handpiece and at least one computer unit, the handpiece and the computer unit are electrically insulated from one another.

### BACKGROUND OF THE INVENTION

### Field of the Invention

The invention relates to a device for operating an intraoral scanner with at least one handpiece and at least one computer unit.

### Description of the Related Art

In dentistry, it is necessary for many tasks to have a model of the teeth and also other intraoral structures. If, in the past, these models were still produced physically via impressions and were stored, for example, as plaster casts, it is now more and more conventional to acquire and store these models virtually. Thus, not only space for storing the casts can be reduced, but the patient is spared the unpleasant taking of the impression.

With respect to their hardware, intraoral scanners have two main units: handpiece and computer unit. Here, it has been shown that the two units are connected using a physical link for communication, since the in part very large amounts of data that can arise during scanning can be communicated at the desired speed. While the computer unit can theoretically be located anywhere, the handpiece inevitably comes in contact with the patient whose virtual impression is to be taken.

When electrically-operated devices are used in the medical field, special measures must be taken to protect the user (MOOP, “Means of Operator Protection”) or the patient (MOPP, “Means of Patient Protection”), the protection of the patient automatically including the protection of the user. These protective measures are conventionally taken by special power supply units that are in any case very expensive for devices with high power consumption and thus greatly increase the costs for the entire device.

### SUMMARY OF THE INVENTION

The object of the invention is therefore to overcome these disadvantages and to make available a cost-favorable method to be able to safely operate intraoral scanners for users and patients.

This object is achieved according to the invention by a device of the initially-named type, which is characterized in that the handpiece and the computer unit are electrically insulated from one another.

The electrical insulation of the handpiece and the computer unit makes it possible to use different power sources for the computer unit and the handpiece. Therefore, the handpiece preferably has its own power source. This is preferably its own power supply unit. Since the handpiece of an intraoral scanner itself conventionally has only a very small power consumption, for this purpose a simple and economical power pack for application in a medical environment can be used.

Alternatively, the handpiece can also be equipped with an energy storage as a power source. For this purpose, there can be a charging station in which the handpiece is stored when it is not being used.

In order to enable data transmission between the two units of the intraoral scanner in spite of the electrical insulation of the handpiece and the computer unit, according to one preferred embodiment of the invention, it is provided that the handpiece and the computer unit are connected using a non-galvanic link for data transmission.

The non-galvanic data transmission link is preferably an optical fiber, since especially high data transmission rates can be achieved this way.

In one preferred embodiment of the invention, the data transmission link has at least two component regions. In one preferred development, at least one component section is a non-galvanic link. According to another preferred development of the invention, it can be in particular an optical fiber.

By dividing the optical fiber into preferably successive component sections or segments, the different advantages of possible transmission paths and links can be used. Thus, for example, electrical insulation can be implemented via an optical fiber, but for another component region, the greater flexibility and the more variable possible applications of metallic conductors can be used.

Furthermore, optical or even electrically conductive lines are often installed in dental treatment chairs. A combination of different and optionally different types of sections or component segments makes it possible for the device to be easily integrated into such a treatment chair.

According to preferred developments of the invention, it is correspondingly provided that at least one component region of the link or even the computer unit is part of a dental treatment chair.

### BRIEF DESCRIPTION OF THE DRAWINGS

Other preferred embodiments of the invention are the subject matter of the dependent claims. One preferred embodiment of the invention that, however, does not limit the protective scope of the application is presented in detail below with reference to the drawing. It shows:

FIG. 1 shows schematically one possible setup of a device according to the invention.

### DESCRIPTION OF THE PREFERRED EMBODIMENTS

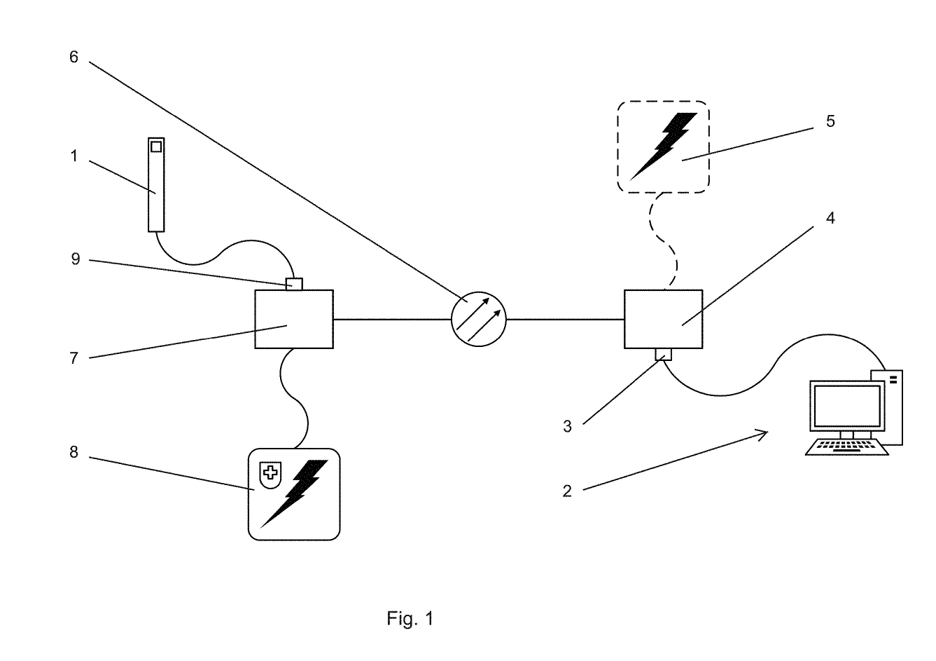

The device that is shown in FIG. 1 for operating an intraoral scanner has a handpiece 1 and a computer unit 2.

In the illustrated embodiment, a computer-side USB link 3 goes from the computer unit 2 to a computer-side transceiver 4. Of course, other links for communication with a computer are also conceivable; for the operation of intraoral scanners, the use of a USB 3.0 interface has in any case proven itself since it can offer not only the desired communications bandwidth, but also an adequate power supply. If a data link should be chosen that is not able to make available an adequate power supply, optionally a power supply unit 5 can be connected in addition to the computer-side transceiver 4. For this purpose, a commercial power supply unit without special safety precautions intended for use in the medical field can be used.

In the computer-side transceiver 4, the control signals coming from the computer unit 2 can then be converted into optical signals 6 and can be relayed via an optical link to a second handpiece-side transceiver 7.

The handpiece-side transceiver 7 is supplied with power from a safety power supply unit 8 that is protected (MOOP) and is suitable for use in a medical context. In the handpiece-side transceiver 7, the optical signal 6 is received and converted again into an electrical signal. In doing so, for example, a USB signal can be generated by the combining data and power supply. In the illustrated embodiment, the combined signal is transmitted via a handpiece-side USB connection 9. The latter is also used to supply the handpiece with power.

The device according to the invention makes it possible to use a computer unit 2 and a handpiece 1 according to the state of the art without they themselves having to be modified for electrically safe use in a medical context.

Another advantage of the device according to the invention arises when an intraoral scanner is to be integrated into an existing treatment chair since, in this way, only space for the safety power supply unit 8, the handpiece-side transceiver 7 and the cabling is required, and the computer unit 2 and optionally also the computer-side transceiver can be positioned spatially separated.

### REFERENCE NUMBER LIST

1 handpiece
2 computer unit
3 USB (computer-side)
4 transceiver (computer-side)
5 power supply unit (normal)
6 optical signal(s)
7 transceiver (handpiece-side)
8 safety power supply unit
9 USB (handpiece-side)

1. Device for operating an intraoral scanner with at least one handpiece (1) and at least one computer unit (2), wherein the handpiece (1) and the computer unit (2) are electrically insulated from one another.

2. Device according to claim 1, wherein the handpiece (1) has a power source.

3. Device according to claim 2, wherein the power source is a safety power supply unit (8).

4. Device according to claim 2, wherein the power source is an energy storage.

5. Device according to claim 1, wherein the handpiece (1) and the computer unit (2) are connected using a non-galvanic link for data transmission.

6. Device according to claim 5, wherein the data transmission link is an optical fiber.

7. Device according to claim 5, wherein the data transmission link has at least two component sections.

8. Device according to claim 7, wherein at least one component section is a non-galvanic link.

9. Device according to claim 7, wherein at least one component section is an optical fiber.

10. Device according to claim 7, wherein at least one component section is part of a dental treatment chair.

11. Device according to claim 1, wherein the computer unit (2) is part of a dental treatment chair.

12. Device according to claim 2, wherein the handpiece (1) and the computer unit (2) are connected using a non-galvanic link for data transmission.

13. Device according to claim 3, wherein the handpiece (1) and the computer unit (2) are connected using a non-galvanic link for data transmission.

14. Device according to claim 4, wherein the handpiece (1) and the computer unit (2) are connected using a non-galvanic link for data transmission.

15. Device according to claim 6, wherein the data transmission link has at least two component sections.

16. Device according to claim 8, wherein at least one component section is an optical fiber.

17. Device according to claim 8, wherein at least one component section is part of a dental treatment chair.

18. Device according to claim 9, wherein at least one component section is part of a dental treatment chair.

19. Device according to claim 2, wherein the computer unit (2) is part of a dental treatment chair.

20. Device according to claim 3, wherein the computer unit (2) is part of a dental treatment chair.
